(19) United States
(12) Patent Application Publication
(10) Pub. No.: US 2013/0109643 A1
(43) Pub. Date: May 2, 2013

(54) METABOLIC INHIBITOR AGAINST TUMORS HAVING AN IDH MUTATION

(71) Applicants: Gregory Joseph Riggins, White Hall, MD (US); Meghan Joyce Seltzer, Herndon, VA (US); Takashi Tsukamoto, Ellicott City, MD (US); Chi Van Dang, Penn Valley, PA (US); Ashkan Emadi, Bethesda, MD (US)

(72) Inventors: Gregory Joseph Riggins, White Hall, MD (US); Meghan Joyce Seltzer, Herndon, VA (US); Takashi Tsukamoto, Ellicott City, MD (US); Chi Van Dang, Penn Valley, PA (US); Ashkan Emadi, Bethesda, MD (US)

(73) Assignee: The Johns Hopkins University, Baltimore, MD (US)

(21) Appl. No.: 13/697,097

(22) PCT Filed: May 10, 2011
(86) PCT No.: PCT/US2011/035841

§ 371 Date: Jan. 11, 2013

Related U.S. Application Data
(60) Provisional application No. 61/333,010, filed on May 10, 2010.
(60) Provisional application No. 61/357,674, filed on Jun. 23, 2010.
(60) Provisional application No. 61/383,426, filed on Sep. 16, 2010.

(51) Int. Cl. A61K 31/433; A61K 31/704

(52) U.S. Cl. CPC A61K 31/433; A61K 31/704
USPC 514/34; 514/363; 435/375; 435/18

(57) ABSTRACT

Methods for treating a cancer having a mutant isocitrate dehydrogenase (IDH), including, but not limited to, a malignant low-grade glioma, a secondary glioblastoma, a transforming myeloproliferative disorder (tMPD), and an acute myelogenous leukemia (AML), with a glutaminase inhibitor, including, bis-2-(5-phenylacetamido-1,2,4-thiadiazol-2-yl)ethyl sulfide (BPTES), are disclosed.

### CROSS-REFERENCE TO RELATED APPLICATIONS

This application claims the benefit of U.S. Provisional Application Nos. 61/333,010, filed May 10, 2010; 61/357,674, filed Jun. 23, 2010; and 61/383,426, filed Sep. 16, 2010, each of which is incorporated herein by reference in its entirety.

### FEDERALLY SPONSORED RESEARCH OR DEVELOPMENT

This invention was made in part with United States Government support under RO1 NS052507, RO1 CA57341, RO1 CA051497, and R21NS074151 awarded by the National Institutes of Health (NIH). The U.S. Government has certain rights in the invention.

### BACKGROUND

Despite the availability of oxygen, cancer cells exhibit high glycolytic rates and increased lactate production, known as the Warburg effect or aerobic glycolysis, rather than high oxidative phosphorylation. Over the past decade, oncogenes (MYC, PI3K, RAS, and AKT) and tumor suppressors (VHL and p53) have been documented to reprogram cancer cell metabolism to aerobic glycolysis. Kim J W., Dana C. V., Cancer's molecular sweet tooth and the Warburg effect. Cancer Res 2006; 66:8927-30; Tennant D. A., et al., Targeting metabolic transformation far cancer therapy. Nat Rev Cancer 2010; 10:267-77. Cancer cells also consume glutamine for energy or as a carbon skeleton or nitrogen donor. DeBerardinis R. J., Cheng T. Q's next; the diverse functions of glutamine in metabolism, cell biology and cancer. Oncogene 2010; 29:313-24; Medina M, A., Glutamine and cancer, J Nutr 2001; 131:2539-42S. Recently, the oncogene MYC was found to induce mitochondrial biogenesis and increase glutamine metabolism, indicating that MYC stimulates both aerobic glycolysis and glutamine oxidation. Gao P., of al., c-Myc suppression of miR-23a/b enhances mitochondrial glutaminase expression and glutamine metabolism. Nature 2009; 458:762-5.

Before the discovery of isocitrate dehydrogenase 1 (IDH1) and 2 (IDH2) mutations, succinate dehydrogenase and fumarate hydratase mutations, which cause hypoxia-inducible factor 1, alpha subunit stabilization and hereditary cancer syndromes, were the only known oncogenic mutations of metabolic enzymes. IDH1 and IDH2 mutations occur frequently in malignant low-grade gliomas, secondary glioblastomas, and acute myelogenous leukemias (AML). Tennant D. A., et. al., Targeting metabolic transformation for cancer therapy. Nat Rev Cancer 2010; 10:267-77; Parsons D. W., et al., An integrated genomic analysis of human glioblastoma multiforme. Science 2008; 321:1807-12; Yan H., et al. IDH1 and IDH2 mutations in gliomas. N. Engl J Med 2009; 360:765-73; Mardis E. R., et al. Recurring mutations found by sequencing an acute myeloid leukemia genome, N Engl J Med 2009; 361:1058-66. These discoveries underscore the importance of metabolic alterations in oncogenesis and suggest the possibility of targeting genetically altered cancer metabolism.

Although wild-type (WT) IDH1 converts isocitrate and NADP+ to α-ketoglutarate (α-KG) and NADPH, mutated amino acids in IDH1 and IDH2 reside in the catalytic pocket and result in a neoenzymatic activity:

α-KG+NADPH→D-2-hydroxyglutarate (2-HG)+NADP⁺.

See Dang L, White D W, Gross S, et al., Cancer-associated IDH1 mutations produce 2-hydroxyglutarate. Nature 2009; 462:739-44. IDH1 mutations in gliomas and AML have, thus far, only been found at residue R132, which is most commonly mutated to a histidine. Yan H., et al. IDH1 and IDH2 mutations in gliomas. N Engl J Med 2009; 360:765-73. Mutations of IDH2 have been found at both R140 and R172. Ward P. S. et al., The common feature of leukemia-associated IDH1 and IDH2 mutations is a neomorphic enzyme activity converting α-ketoglutarate to 2-hydroxyglutarate. Cancer Cell 2010; 17:225-34. All documented IDH1 or IDH2 mutations result in the ability to produce 2-HG from α-KG. Dang L., et al., Cancer-associated IDH1 mutations produce 2-hydroxyglutarate. Nature 2009; 462:739-44; Ward P. S., et al., The common feature of leukemia-associated IDH1 and IDH2 mutations is a neomorphic enzyme activity converting α-ketoglutarate to 2-hydroxyglutarate. Cancer Cell 2010; 17:225-34.

Although the role of IDH1 mutation in tumorigenesis has not been determined, changes in enzymatic function that result from IDH1 mutation likely contribute to tumor formation. Decreased NADPH production from loss of IDH1 WT function coupled with increased 2-HG levels could lead to oxidative stress. Bleeker F. E., et al., The prognostic IDH1 (R132) mutation is associated with reduced NADP+-dependent IDH activity in glioblastoma. Acta Neuropathol 2010; 119:487-94; Latini A., et al., D-2-hydroxyglutaric acid induces oxidative stress in cerebral cortex of young rats. Eur Neurosci 2003; 17:2017-22. Secondly, 2-HG interferes with the electron transport chain and could alter mitochondrial physiology and drive cells toward aerobic glycolysis. Latini A., et al., Mitochondrial energy metabolism is markedly impaired by D-2-hydroxyglutaric acid in rat tissues. Mol Genet Metab 2005; 86:188-99. Due to structural similarity between 2-HG and α-KG, 2-HG also could interfere with the function of enzymes that utilize α-KG (e.g., histone demethylases). Lastly, 2-HG is produced in inborn errors of metabolism (L-2- or D-2-hydroxyglutaric aciduria) where the enzyme that metabolizes 2-HG (L- or D-2-hydroxyglutarate dehydrogenase) is nonfunctional. Struys E. A., et al., Mutations in the D-2-hydroxyglutarate dehydrogenase gene cause D-2-hydroxyglutaric aciduria. Am J Hum Genet. 2005; 76:358-60; Rzem R., et al., L-2-hydroxyglutaric aciduria, a defect of metabolite repair. J inherit Moab Dis 2007; 30:681-9. Further, individuals with L-2-hydroxyglutaric aciduria have been documented to develop gliomas, Aghili M., et al., Hydroxyglutaric aciduria and malignant brain tumor: a case report and literature review. J Neurooncol 2009; 91:233-6, but not those with D-2-hydroxyglutaratic aciduria (the enantiomer produced by mutant IDH1/2).

It is not clear if blocking IDH1 mutant activity would be an effective therapy, particularly if the mutant protein is only involved in tumor initiation. Because mutant IDH1 (mIDH1) tumors require α-KG to produce 2-HG, however, they could consequently be susceptible to alteration of α-KG homeostasis. It has been shown that 2-HG is primarily derived from glutamine. See FIG. 1; Dang L., et al., Cancer-associated IDH1 mutations produce 2-hydroxyglutarate. Nature 2009; 462:739-44. Glutamine is hydrolyzed by glutaminase to produce glutamate that is subsequently converted to α-KG. α-KG is then converted to 2-HG by mutant IDH1. Whether inhibiting glutaminase might perturb α-KG homeostasis and yield a selective response in cancer cells bearing IDH1 mutant enzymes has yet to be determined.

### SUMMARY

In one aspect, the presently disclosed subject matter provides a method for treating a cancer having a mutant isocitrate dehydrogenase (IDH) in a subject in need of treatment thereof, the method comprising administering to the subject a therapeutically effective amount of a glutaminase inhibitor.

In some aspects, the glutaminase inhibitor is selected from the group consisting of:

(a) a compound of Formula (I):

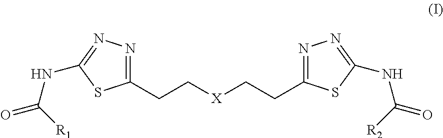

wherein: X is sulfur or oxygen; R₁ and R₂ are independently selected from the group consisting of lower alkyl, lower alkoxyl, aryl, thiophenyl and —(CH₂)ₙ-aryl; wherein n is 0 or 1, and aryl is a monocyclic aromatic or heteroaromatic group, having ring atoms selected from the group consisting of carbon, nitrogen, oxygen, and sulfur, and having at most three non-carbon ring atoms, which group may be unsubstituted or substituted with one or more substituents selected from the group consisting of halogen, lower alkyl, lower alkoxyl, amino, lower alkyl amino, amino(lower alkyl), or halo(lower alkyl); and

(b) a compound selected from the group consisting of 6-diazo-5-oxo-L-norleucine, acivicin, N-ethylmaleimide, p-chloromercuriphenylsulfonate, L-2-amino-4-oxo-5-chloropentoic acid, azaserine, and 5-(3-bromo-4-(dimethylamino)phenyl)-2,2-dimethyl-2,3,5,6-tetrahydrobenzo[a]phenanthridin-4(1H)-one; and pharmaceutically acceptable salts thereof. In certain aspects, the compound of Formula (I) is bis-2-(5-phenylacetamido-1,2,4-thiadiazol-2-yl)ethyl sulfide (BPTES).

In particular aspects, the IDH mutation is selected from an IDH1 mutation and an IDH2 mutation. Representative cancers treatable by the presently disclosed subject matter include, but are not limited to, a malignant low-grade glioma, a secondary glioblastoma, a transforming myeloproliferative disorder (tMPD), and an acute myelogenous leukemia (AML). In other aspects, the presently disclosed subject matter further comprises administering a glutaminase inhibitor in combination with one or more of an antimetabolite, an anthracycline, and combinations thereof.

In other aspects, the presently disclosed subject matter provides a method for selectively inhibiting growth of a cell expressing mutant isocitrate dehydrogenase (IDH), the method comprising administering to the cell an effective amount of a glutaminase inhibitor. In particular aspects, the glutaminase inhibitor is a compound of Formula (I), as defined hereinabove. In other aspects, the glutaminase inhibitor is a compound selected from the group consisting of 6-diazo-5-oxo-L-norleucine, acivicin, N-ethylmaleimide, p-chloromercuriphenylsulfonate, L-2-amino-4-oxo-5-chloropentoic acid, azaserine, and 5-(3-bromo-4-(dimethylamino)phenyl)-2,2-dimethyl-2,3,5,6-tetrahydrobenzo[a]phenanthridin-4(1H)-one. In some aspects, the cell is a cancer cell. In particular aspects, the mutant IDH is selected from the group consisting of a mutant IDH1 and a mutant IDH2.

In further aspects, the presently disclosed subject matter provides a method for selecting or stratifying a subject for treatment with a glutaminase inhibitor, the method comprising measuring a 2-hydroxyglutarate (2-HG) level in a biological sample, e.g., serum, from the subject and selecting the subject for treatment if the 2-HG level exceeds a predetermined threshold value.

In other aspects, the presently disclosed subject matter provides a method for selecting a subject for treatment with a glutaminase inhibitor, the method comprising identifying the presence of an IDH1 or IDH2 mutation in a cell or tissue from the subject.

In yet other aspects, the presently disclosed subject matter provides a method for monitoring a treatment of a subject with a glutaminase inhibitor, the method comprising measuring a 2-HG level in a biological sample from the subject and comparing the 2-HG level in the subject with a 2-HG level selected from the group consisting of a previous biological sample from the subject, a predetermined threshold value of 2-HG, a 2-HG level in one or more biological samples from a control group, and combinations thereof.

In yet further aspects, the presently disclosed subject matter provides a method for detecting transformation in a subject afflicted with a myeloproliferative disorder (MPD), the method comprising measuring a 2-HG level in a biological sample, e.g., serum, from the subject and comparing the 2-HG level with a predetermined threshold value to detect transformation. In particular aspects, the method further comprises prophylactic treatment of the subject with a glutaminase inhibitor to delay or prevent the onset of the transformation.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 is a pathway showing production of 2-HG from glutamine and inhibitor targets. Legend: GLS, glutaminase; GOT, glutatamate oxaloacetate transaminase; GLUD, glutamate dehydrogenase; IDH1, isocitrate dehydrogenase; IDH1ₘᵤₜ, mutant IDH1;

FIGS. 2A-2C show the validation of tet-inducible, stable D54 glioblastoma lines. FIG. 2A, Western blot showing doxycyline (Dox)-induced expression of 6×-His-Tag-IDH1. FIG. 2B, 2-hydroxyglutarate liquid chromatography/mass spectrometry (LC/MS) retention peaks. FIG. 2C, 2-HG and α-KG levels measured by LC/MS;

FIG. 3 shows conversion of isocitrate to α-KG in cell lysates from cells that overexpress WT or R132H IDH1 or the parental cell line was measured by quantifying Δ340 nm as protein amounts were increased;

FIGS. 4A and 4B demonstrate that mutant IDH1 cells depend on glutaminase for cell growth and glutaminase inhibition is negated by dimethyl-α-ketoglutarate. FIG. 4A, anti-glutaminase siRNA (siGLS) slows growth of mutant IDH1 cells. Western blot shows decreased levels of glutaminase in response to siGLS. FIG. 4B, effects of BPTES in the absence or presence of 1 mmol/L dimethyl-α-ketoglutarate (dmαKG) were measured. Data are from one representative experiment of three with similar trends and the average and SE of four replicates at each concentration. *, P≦0.05. For FIG. 4A, P value is for siGLS compared with siCont. Cell number was normalized to day 0. For FIG. 4B, the P value was for D54+R132H compared with D54 and D54+WT IDH1. Fold growth represents the ratio of alamarBlue fluorescence units of treated cells to vehicle-treated cells;

FIG. 5 demonstrates that mutant IDH1 cells are dependent on glutaminase for survival and the effect of glutaminase inhibition is negated by dimethyl-α-ketoglutarate (dmαKG). The effect of BPTES in the absence or presence of 1 mM dimethyl-α-ketoglutarate was measured on cells that overexpress either mutant or WT IDH1. Shown here is one representative experiment of three with similar trends, and the average and SEM of four replicates at each concentration. * corresponds to p-value ≦0.05. The p-value was for D54+R132H IDH1+Dox compared to all other lines with and without doxycycline. Fold growth represents the ratio of alamarBlue fluorescence units of treated cells to that of vehicle treated cells;

FIGS. 6A and 6B show that BPTES does not induce apoptosis in mutant IDH1 cells: (A) Caspase 3/7 induction does not increase in BPTES treated cells. Fold induction of caspase 3/7 is expressed as luminescence of BPTES treated cells divided by the luminescence of DMSO treated cells; (B) Annexin V staining does not increase in BPTES treated cells. Percentages shown in the figure represent the percentage of gated events in the quadrant;

FIG. 7 shows that dependence of glutaminase is not limited to D54 background. TNA, which constitutively express either wildtype or mutant IDH1, were exposed to BPTES and the effect of cell growth was measured. Shown here is one representative experiment of three with similar trends, and the average and SEM of four replicates at each concentration. * corresponds to p-value ≦0.05. The p-value was for GT+R132H compared to GT and GT+WT IDH1. Fold growth represents the ratio of alamarBlue fluorescence units of treated cells to that of vehicle treated cells;

FIGS. 8A-8C demonstrate that metabolic changes result from overexpression of mutant IDH1 and 48 hours of treatment with 10 mmol/L BPTES. Glutaminase activity (A) and glutamate, α-KG, and 2-HG levels (B) were measured in WT and mutant IDH1 cells. In FIG. 8C, levels of other metabolites measured using LC/MS in response to BPTES treatment. *, P≦0.05 for 4B and 4C. The P value is for either D54 WT IDH1 DMSO versus 10 mmol/L BPTES or D54 R132H IDH1 DMSO versus 10 mmol/L BPTES;

FIG. 9 is a heat map showing metabolic alterations in mutant and WT IDH1 cells with and without 10 nM BPTES treatment. Black corresponds to median value of untreated WTs2 IDH1 cells. The scale is log 2 with yellow representing increases in metabolites and cyan representing a decrease. QQQ signifies that the data were from a triple quadrupole mass spectrometer. All other data are from an exact mass Exactive instrument;

FIGS. 10A and 10B show that cells overexpressing mutant IDH1 are susceptible to treatment with EGCG and AOA. The effect of EGCG (A) or AOA (B) was measured on cells that overexpress either mutant or WT IDH1. EGCG experiments were conducted with 0.1 g/L glucose, and AOA experiments were conducted with 1.5 g/L glucose. Shown here is one representative experiment of three with similar trends, and the average and SEM of four replicates at each concentration. * corresponds to p-value ≦0.05. The p-value was for D54+R132H IDH1+Dox compared to all other lines with and without doxycycline. Fold growth represents the ratio of alamarBlue fluorescence units of treated cells to vehicle treated cells;

FIG. 11 shows that cells expressing mutant IDH1 are susceptible to targeting of a specific pathway rather than alteration of glutamine levels. Cells were exposed to different concentrations of glutamine, and cell viability was measured. Shown here is one representative experiment of three with similar trends, and the average and SEM of four replicates at each concentration. Fold growth represents the ratio of fluorescence at 485 nm for cells at the indicated glutamine concentration to that of cells at 4 mM glutamine; and

FIGS. 12A-12F shows growth curves of primary AML cells (with mutational status indicated) in response to control DMSO vehicle or BPTES at 20 or 40 μM.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. METABOLIC INHIBITORS AGAINST CANCERS HAVING AN IDH MUTATION

Isocitrate dehydrogenases, IDH1 and IDH2, catalyze the conversion of isocitrate to α-ketoglutarate with the production of NADPH:

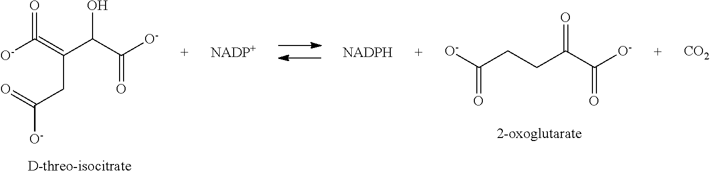

Mutants of IDH1 and IDH2 found in gliomas and acute myelogenous leukemia (AML), however, convert α-ketoglutarate (2-oxoglutarate) to 2-hydroxyglutarate (2-HG) with the consumption of NADPH:

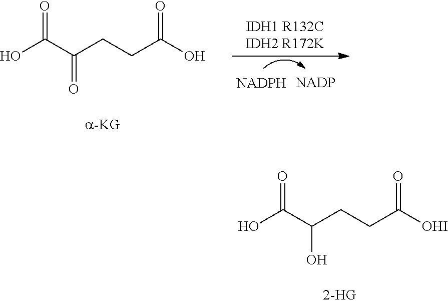

The primary source for α-ketoglutarate under this condition is glutamine, which is converted to glutamate by glutaminase and then to α-ketoglutarate. Because glutamine is the primary source for α-ketoglutarate, it is thought that cells with IDH mutations are dependent on glutamine, which replenishes α-ketoglutarate that is siphoned by the mutant enzyme. As such, the mIDH expressing cells are in essence addicted to glutamine via glutaminase. Whether IDH mutations are driver mutations or those that are involved in tumor maintenance does not affect the fact that these cells become addicted to glutamine, such that depletion of glutamine or interruption of its metabolism would be detrimental to cells expressing mIDH.

More particularly, mutation at the R132 residue of isocitrate dehydrogenase 1 (IDH1), frequently found in gliomas and acute myelogenous leukemia, creates a neoenzyme that produces 2-hydroxyglutarate (2-HG) from α-ketoglutarate (α-KG). In some embodiments, the presently disclosed subject matter therapeutically exploits this neoreaction in mutant IDH1 cells that require α-KG derived from glutamine Glutamine is converted to glutamate by glutaminase and further metabolized to α-KG.

The presently disclosed subject matter, in some embodiments, demonstrates that glutaminase can be inhibited with siRNA or a glutaminase inhibitor, which in some embodiments includes the small molecule inhibitor bis-2-(5-phenylacetamido-1,2,4-thiadiazol-2-yl)ethyl sulfide (BPTES), which slowed growth of glioblastoma cells expressing mutant IDH1 compared with those expressing wild-type IDH1. Growth suppression of mutant IDH1 cells by BPTES was rescued by adding exogenous α-KG. BPTES inhibited glutaminase activity, lowered glutamate and α-KG levels, and increased glycolytic intermediates while leaving total 2-HG levels unaffected. The ability to selectively slow growth in cells with IDH1 mutations by inhibiting glutaminase suggests a unique reprogramming of intermediary metabolism and a potential therapeutic strategy. Accordingly, in some embodiments, the presently disclosed subject matter demonstrates that glutaminase could be a potential therapeutic target in mutant IDH1 cancer cells.

Accordingly, in some embodiments, the presently disclosed subject matter provides a method for treating a cancer having a mutant isocitrate dehydrogenase (IDH) in a subject in need of treatment thereof, the method comprising administering to the subject a therapeutically effective amount of a glutaminase inhibitor. In some embodiments, the glutaminase inhibitor is selected from the group consisting of:

(a) a compound of Formula (I):

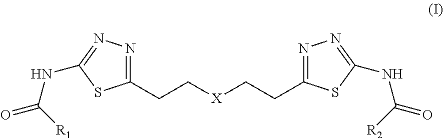

wherein: X is sulfur or oxygen; R₁ and R₂ are independently selected from the group consisting of lower alkyl, lower alkoxyl, aryl, thiophenyl and —(CH₂)ₙ-aryl; wherein n is 0 or 1, and aryl is a monocyclic aromatic or heteroaromatic group, having ring atoms selected from the group consisting of carbon, nitrogen, oxygen, and sulfur, and having at most three non-carbon ring atoms, which group may be unsubstituted or substituted with one or more substituents selected from the group consisting of halogen, lower alkyl, lower alkoxyl, amino, lower alkyl amino, amino(lower alkyl), or halo(lower alkyl); and

(b) a compound selected from the group consisting of 6-diazo-5-oxo-L-norleucine, acivicin, N-ethylmaleimide, p-chloromercuriphenylsulfonate, L-2-amino-4-oxo-5-chloropentoic acid, azaserine, and 5-(3-bromo-4-(dimethylamino)phenyl)-2,2-dimethyl-2,3,5,6-tetrahydrobenzo[a]phenanthridin-4(1H)-one; and pharmaceutically acceptable salts thereof.

Representative compounds of Formula (I) include such compounds disclosed in U.S. Pat. No. 6,451,828 to Newcomb, which is incorporated herein by reference in its entirety. In some embodiments of Formula (I), X is sulfur. In particular embodiments, each of R₁ and R₂ is selected from the group consisting of —(CH₂)ₙ-aryl, 2-thiophenyl, 2-furanyl, phenyl or benzyl, unsubstituted or substituted with lower alkyl or lower alkoxyl, benzyl, p-methoxy phenyl, R₁ and R₂ is m-tolyl, lower alkoxyl, ethoxy, lower alkyl, and t-butyl. In particular embodiments, the compound of Formula (I) is bis-2-(5-phenylacetamido-1,2,4-thiadiazoi-2-yl)ethyl sulfide (BPTES):

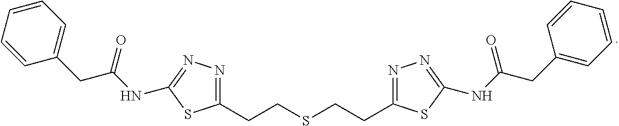 [SMILES: O=C(Cc1ccccc1)Nc1nnc(CCSCCc2nnc(NC(=O)Cc3ccccc3)s2)s1 ; "."]

The cancer treatable by the presently disclosed subject matter includes any cancer, e.g. solid tumor or a cancer derived from hematopoietic (blood-forming) cells, which is associated with an IDH mutation, including an IDH1 mutation or an IDH2 mutation. Representative cancers include, but are not limited to, a malignant low-grade glioma, a secondary glioblastoma, a transforming myeloproliferative disorder (tMPD), and an acute myelogenous leukemia (AML).

In some embodiments, as disclosed in more detail herein below, the method further comprises administering a glutaminase inhibitor in combination with one or more of an antimetabolite, an anthracycline, and combinations thereof.

In other embodiments, the presently disclosed subject matter provides a method for selectively inhibiting growth of a cell expressing mutant isocitrate dehydrogenase (IDH), the method comprising administering to the cell an effective amount of a glutaminase inhibitor. In some embodiments, the glutaminase inhibitor is a compound of Formula (I), as defined hereinabove. In other embodiments, the glutaminase inhibitor is a compound selected from the group consisting of 6-diazo-5-oxo-L-norleucine, acivicin, N-ethylmaleimide, p-chloromercuriphenylsulfonate, L-2-amino-4-oxo-5-chloropentoic acid, azaserine, and 5-(3-bromo-4-(dimethylamino)phenyl)-2,2-dimethyl-2,3,5,6-tetrahydrobenzo[a]phenanthridin-4(1H)-one. The mutant IDH is selected from the group consisting of a mutant IDH1 and a mutant IDH2. In particular embodiments, the cell comprises a cancer cell. In representative embodiments, the cancer cell is selected from the group consisting of a malignant low-grade glioma, a secondary glioblastoma, and an acute myelogenous leukemia (AML).

### II. INHIBITION OF GLUTAMINASE PREFERENTIALLY SLOWS GROWTH OF GLIOMA CELLS WITH MUTANT IDH1

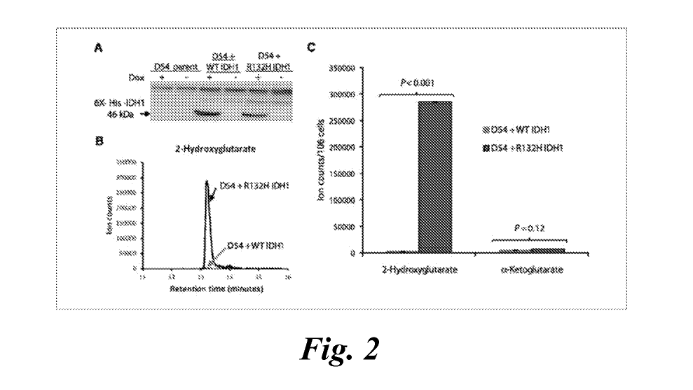

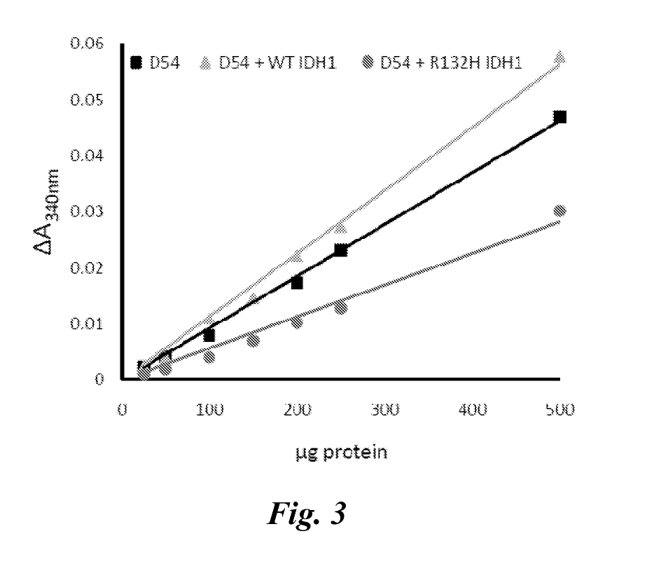

Currently, no established glioblastoma cell lines are reported to possess IDH1 or IDH2 mutations and attempts to derive cell lines from patients with an IDH1 mutation have been unsuccessful. Therefore, tet-inducible, stable D54 glioblastoma cell lines that overexpress WT or R132H IDH1 were created (FIG. 2A). Expression of R132H IDH1 decreased total IDH activity by 50% and 25% compared with cells overexpressing WT IDH1 and parental D54 cells, respectively (FIG. 3). This observation corroborates decreased IDH activity in cell culture models and glioblastoma tumor sections. Yan F L, et al., IDH1 and IDH2 mutations in gliomas. Engl. J Med 2009; 360:765-73; Bleeker F. E, et al., The prognostic IDH1 (R132) mutation is associated with reduced NADP+-dependent IDH activity in glioblastoma. Acta Neuropathol 2010; 119:487-94; Zhao S., et al. Glioma-derived mutations in IDH1 dominantly inhibit IDH1 catalytic activity and induce HIF-1α. Science 2009; 324:261-5. 2-HG levels were elevated in mutant IDH1 cells compared with WT IDH1-expressing cells, whereas α-KG levels were not significantly different (FIGS. 2B and 2C).

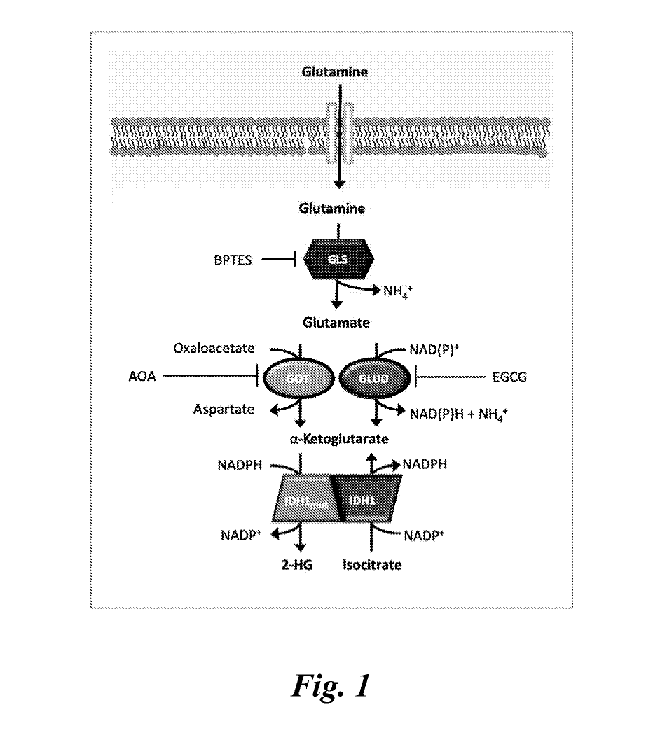

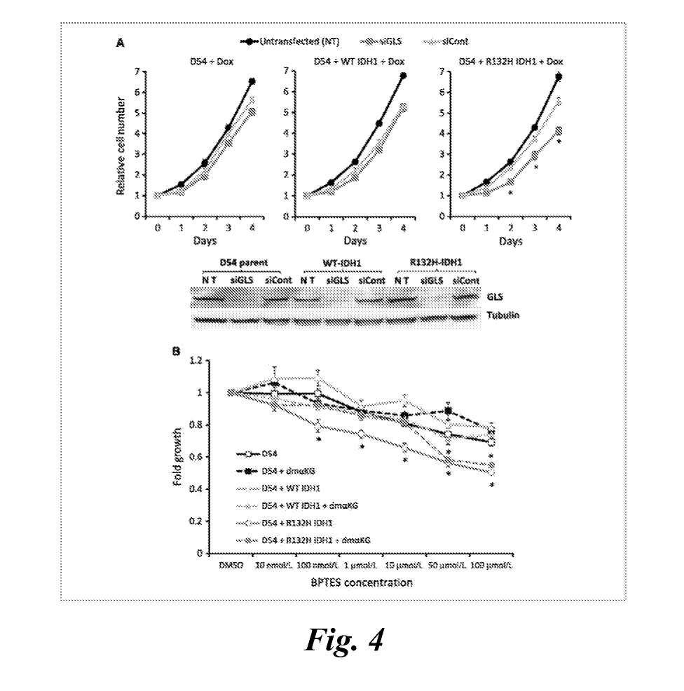

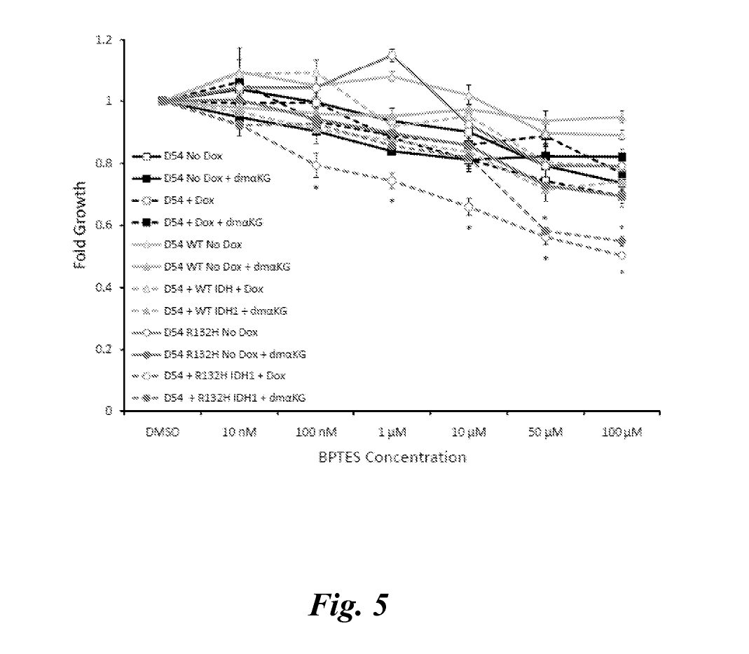

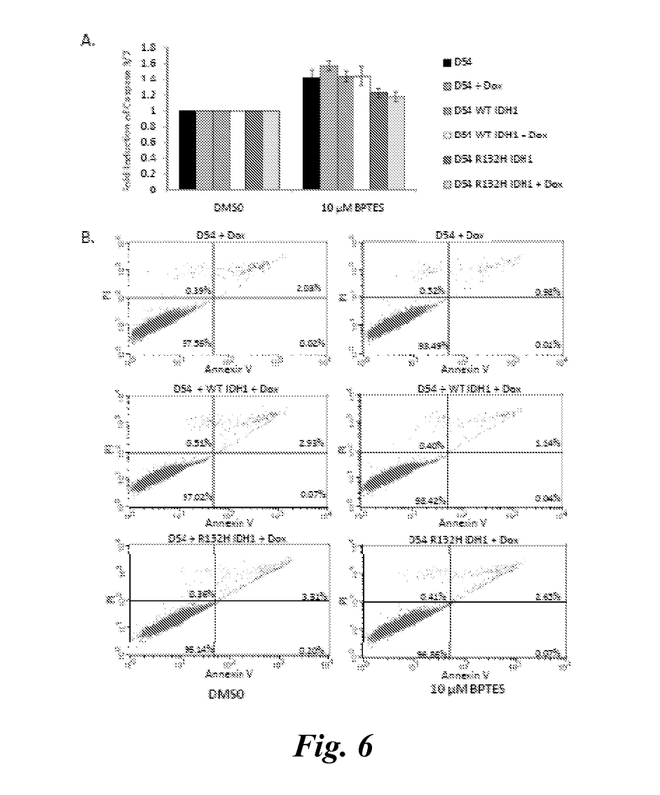

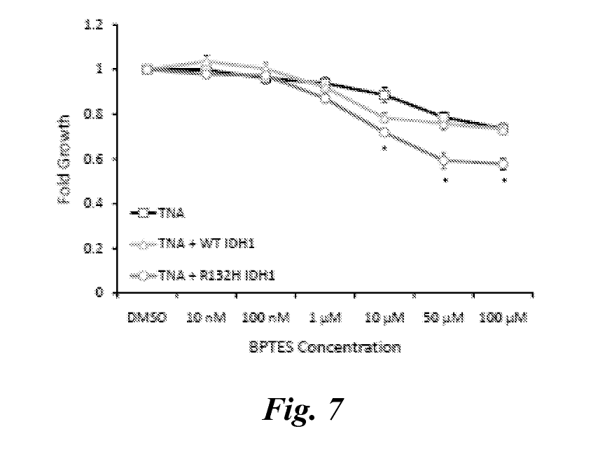

Because previous studies had shown that α-KG consumed by mutant IDH1 was derived from glutamine (see FIG. 1; Dang L., et al., Cancer-associated IDH1 mutations produce 2-hydroxyglutarate. Nature 2009; 462:739-44, glutaminase was targeted with siRNA to determine if mutant IDH1 cells exhibited decreased growth compared with WT IDH1 cells. siRNA against glutaminase specifically slowed the growth of mutant IDH1 cells, but not parental cells or cells overexpressing WT IDH1 (FIG. 4A). Consistent with the effects of anti-glutaminase siRNA, BPTES, a glutaminase inhibitor, Robinson M. M., et al., Novel mechanism of inhibition of rat kidney-type glutaminase by bis-2-(5-phenylacetamido-1,2,4-thiadiazol-2-yl)ethyl sulfide (BPTES). Biochem J 2007; 406:407-14, preferentially slowed growth of mutant IDH1 cells without inducing apoptosis (FIG. 4B, FIG. 5, and FIG. 6). Transformed normal human astrocytes (TNA), which overexpressed R132H or WT IDH1, also were treated with BPTES and it was found that mutant IDH1 cells also were more sensitive to BPTES (FIG. 7).

Then, α-KG levels were restored in BPTES-treated cells to determine whether this would ameloriate growth inhibition. Exposing cells to dimethyl-α-ketoglutarate, a cell permeable α-KG precursor, significantly reduced growth inhibition of mutant IDH1 cells by BPTES (FIG. 4B, FIG. 5). These observations suggest that glutaminase inhibition in mutant IDH1 cells decreased α-KG levels, potentially altering intermediary metabolism and consequently inhibiting cell proliferation. The effects of BPTES at higher concentrations, however, were not blocked by 1 mmol/L dimethyl-α-ketoglutarate. Although the reasons for this are unclear, without wishing to be bound to any one particular theory, it is possible that 1 mmol/L dimethyl-α-ketoglutarate is not sufficient to rescue the effects of 50 or 100 mmol/L BPTES, or BPTES may have additional growth inhibitory effects at elevated concentrations.

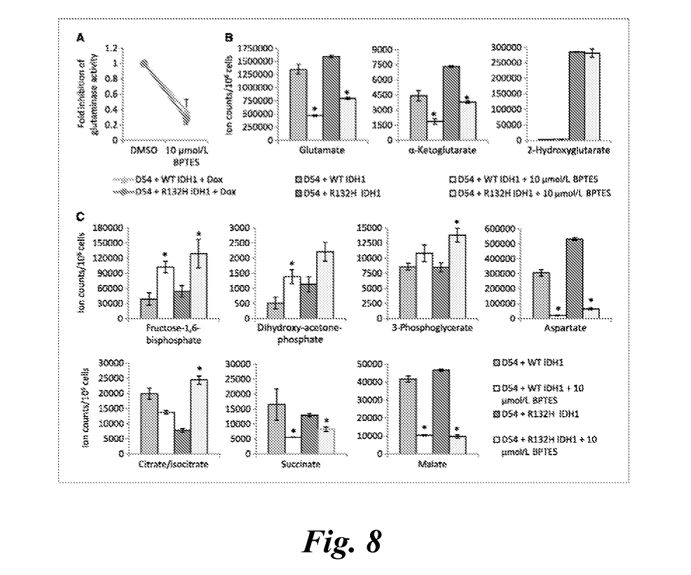

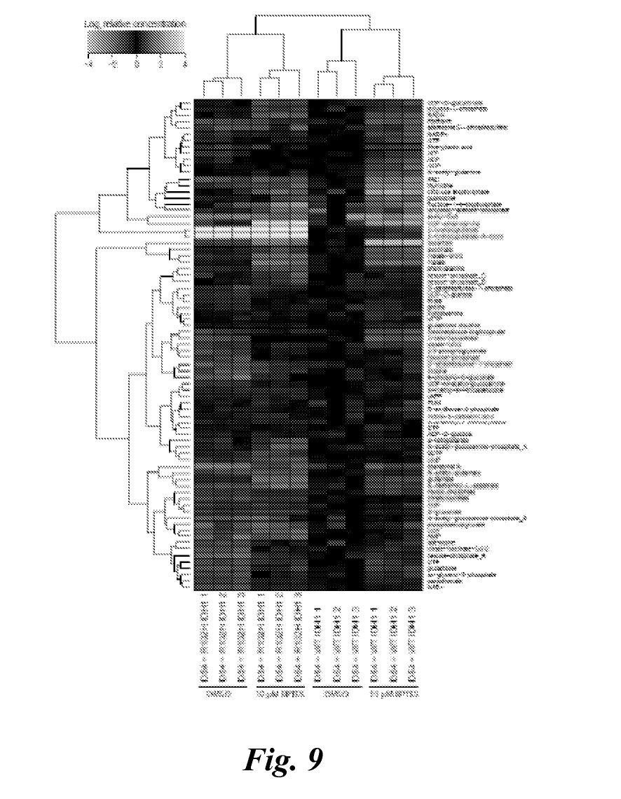

Glutaminase activity was significantly reduced in both WT and mutant IDH1 expressing cells (59% and 68% inhibition, respectively) by 10 mmol/L BPTES (FIG. 8A). Consistent with decreased glutaminase activity, BPTES treatment diminished glutamate and α-KG levels. Lowered α-KG led to decreases in subsequent tricarboxylic acid cycle intermediates (succinate and malate), as well as aspartate, which is derived from transamination of oxaloacetate with glutamate serving as the nitrogen donor. Surprisingly, 2-HG levels remained unchanged in treated mutant IDH1 cells (FIG. 8B, FIG. 9). Levels of glycolytic intermediates (fructose-1,6-bisphosphate, dihydroxy-acetone-phosphate, and 3-phosphoglycerate) increased with BPTES treatment, however, indicating that glycolytic flux is altered. Further, citrate levels changed in diametrically opposite directions between WT and IDH1 mutant cells before and after BPTES treatment. Basal citrate levels were lower, but increased in treated mutant IDH1 cells as compared with higher basal levels in WT IDH1 cells that decreased with BPTES treatment (FIG. 8C).

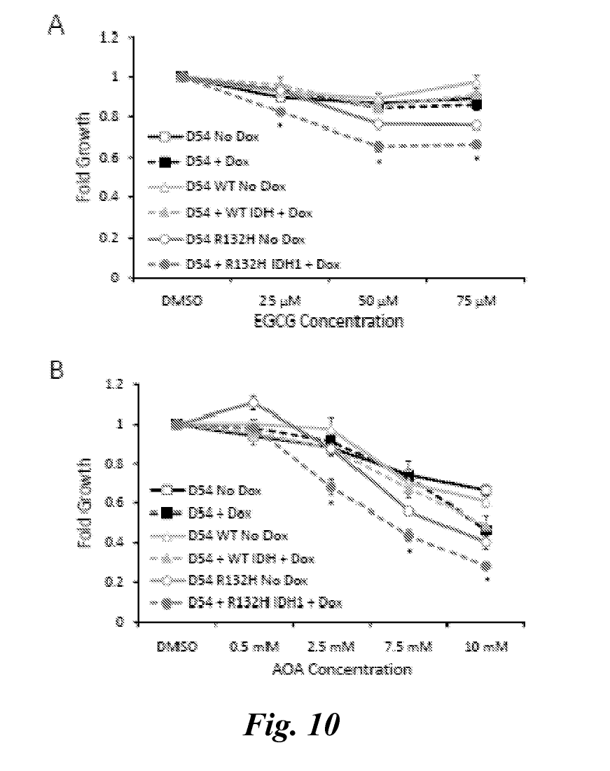

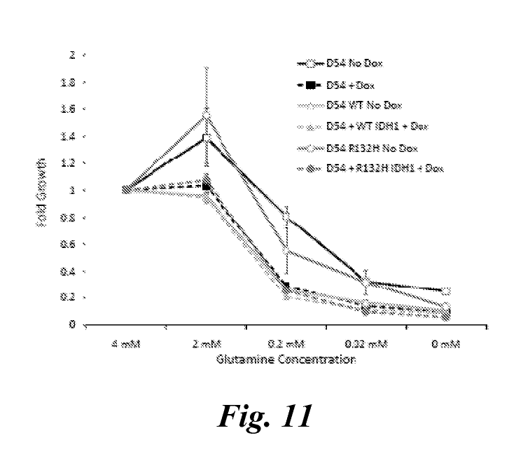

To further show the sensitivity of mutant IDH1 cells to inhibition of α-KG synthesis, enzymes that convert glutamate to α-KG were inhibited. Mutant IDH1 cells were more sensitive to both epigallocatechin gallate (EGCG), a glutamate dehydrogenase inhibitor, and amino-oxyacetic acid (AOA), a pan-transaminase inhibitor. A reduced glucose concentration in the media was required to see the effects of EGCG and AOA, however, suggesting that reduced glycolytic compensation is necessary to unmask the sensitivity of mutant IDH1 cells to these inhibitors (FIG. 10). Mutant IDH1 cells were not more sensitive to glutamine deprivation than WT cells (FIG. 11). Nonetheless, acquisition of mutant IDH1 activity seems sufficient to sensitize cells to inhibition of α-KG synthesis.

Therapies that target various aspects of cancer cell metabolism are currently being developed and are primarily focused on glucose metabolism. Tennant D. A., et al., Targeting metabolic transformation for cancer therapy. Nat Rev Cancer 2010; 10:267-77. The dependence of cancer cells on glutamine for various processes is well documented, Tennant D. A., et al., Targeting metabolic transformation for cancer therapy. Nat Rev Cancer 2010; 10:267-77; DeBerardinis R. J., Cheng T., Q's next: the diverse functions of glutamine in metabolism, cell biology and cancer. Oncogene 2010; 29:313-24; Medina M. A., Glutamine and cancer. J Nutr 2001; 131:2539-42S, and also has been a target of interest for therapy; however, clinical trials have yielded little success due to a lack of efficacy or undesirable side effects of glutamine analogs. Tennant D. A., et al. Targeting metabolic transformation for cancer therapy. Nat Rev Cancer 2010; 10:267-77; Medina. M. A., Glutamine and cancer. J Nutr 2001; 131:2539-42S.

The presently disclosed subject matter provides inhibitors of glutamine metabolism, e.g., BPTES, which allosterically inhibits glutaminase (but not glutaminase 2) and is not a glutamine analog. Robinson M. M., et al., Novel mechanism of inhibition of rat kidney-type glutaminase by bis-2-(5-phenylacetamido-1,2,4-thiadiazol-2-yl)ethyl sulfide (BPTES). Biochem J 2007; 406:407-14. More particularly, the presently disclosed subject matter investigates the dependency of cancer cells with IDH1 mutation on glutaminase activity for maintenance of α-KG homeostasis.

The discovery of IDH1 mutations, Parsons D W, Jones S, Zhang X, et al. An integrated genomic analysis of human glioblastoma multiforme. Science 2008; 321: 180:7-12, identified a metabolic genetic alteration present in a large fraction of gliomas and cytogenetically normal AML. Parsons D. W., et al. An integrated genomic analysis of human glioblastoma multiforme. Science 2008; 321:1807-12; Mardis E. R., et al., Recurring mutations found by sequencing an acute myeloid leukemia genome, N Engl J Med 2009; 361:1058-66. Genetically, a clustering of heterozygous mutations in IDH1 at a single residue indicates a gain-of-function mutation that is supported by the gain of a new enzymatic activity by mutant IDH1. Rather than converting isocitrate to α-KG, mutant IDH1 consumes α-KG and produces 2-HG. Studies have shown that glutamine serves as a cellular source of α-KG consumed by mutant IDH1. Dang L., et al., Cancer-associated IDH1 mutations produce 2-hydroxyglutarate. Nature 2009; 462: 739-44. It is not currently understood whether inhibiting mutant IDH1 and reducing 2-HG production would be therapeutically useful, because a role for mutant IDH1 or 2-HG in tumor maintenance has not been established. Thus, the presently disclosed subject matter provides an approach to slow mutant IDH1 cell growth by inhibiting glutaminase.

The presently disclosed subject matter shows that BPTES inhibits glutaminase activity and consequently lowers glutamate and α-KG levels in mutant and WT IDH1 cells, but only growth of mutant IDH1 cells is preferentially slowed in response to BPTES treatment. BPTES treatment was associated with elevated glycolytic intermediates, which may reflect a compensatory increase in glycolysis to produce α-KG and maintain homeostasis. The notion that glutaminase inhibition perturbs α-KG homeostasis and causes growth inhibition is supported by rescue experiments using a membrane permeable α-KG precursor. Further, inhibition of enzymes that convert glutamate to α-KG showed selectivity for mutant IDH1 cells, but only under glucose-deprived conditions. Mutant IDH1 cells, however, were not more susceptible than WT IDH1 cells to glutamine deprivation, and withdrawing glutamine altogether will eventually slow the growth of any cell that requires glutamine as an essential nutrient. This result raises the possibility that inhibition of glutaminase may have a different therapeutic result on IDH1 mutant cells compared with inhibition of glutamine uptake.

More particularly, the presently disclosed subject matter reveals the following: First, although BPTES treatment lowered glutamate and α-KG levels, 2-HG levels were not significantly decreased. Accordingly, if 2-HG competes with α-KG for binding sites on α-KG-dependent enzymes, occupancy of these sites with α-KG would fall with BPTES treatment. Although the effect likely would not be significant in wild-type cells, where α-KG presumably fills most sites, it could contribute to impaired cell growth upon BPTES treatment of cells expressing mutant IDH1. Second, BPTES treatment decreased subsequent tricarboxylic acid cycle intermediates, succinate and malate. Additionally, levels of glycolytic intermediates were increased, showing that metabolism is perturbed even far from the tricarboxylic acid cycle.

Without wishing to be bound to any one particular theory, it is believed that glycolytic intermediates are increased because of increased glycolytic flux to compensate for lowered α-KG levels; however, the possibility that intermediates build up due to decreased glycolytic flux has not been ruled out. One observation is the diametrically opposite changes in citrate levels between treated WT and R132H IDH1 cells. Currently, the reasons for these differences are not fully understood, and the dissection of these mechanisms is beyond the scope of the present disclosure. Nonetheless, differences in intermediary metabolism between WT and mutant IDH1 cells are sufficient to provoke a gain of sensitivity to glutaminase inhibition in cells with mutant IDH1.

Although reduction of glutaminase activity is significant in mutant IDH1 cells, the effects of BPTES on growth are modest (about 20% growth reduction). The modest growth reduction is not particularly surprising, because D54 cells have a genetic background that can tolerate significant siRNA-mediated reduction of glutaminase level. The simple overexpression of mutant IDH1, however, alters intermediary metabolism sufficiently to sensitize mutant IDH1 cells to glutaminase inhibition. Again, without wishing to be bound to any one particular theory, it is thought that glioma cells with a naturally occurring IDH1 mutation would show increased susceptibility to glutaminase inhibition; however, no such cell lines exist at present.

Additionally, cellular metabolism is incredibly dynamic and seems to compensate for changes in intermediary metabolism, such as increased glycolysis, upon BPTES treatment. As a result, it is thought that glutaminase inhibition will not be effective as a single-arm therapy, but will be a part of a more complex strategy that may involve simultaneous inhibition of glycolysis.

Like all treatments, there could be potential disadvantages to this therapeutic strategy because glioblastoma cells engineered with mutant IDH1 or IDH2 could have a pseudohypoxic phenotype. Zhao S., et al., Glioma-derived mutations in IDH1 dominantly inhibit IDH1 catalytic activity and induce HIF-1a. Science 2009; 324:261-5. Previous studies documented that exogenous α-KG could reactivate prolyl hydroxylase, decrease hypoxia-inducible factor 1, alpha subunit levels, and inhibit cell growth resulting from pseudohypoxia elicited by mutations in succinate dehydrogenase and fumarate hydratase. MacKenzie E. D., et al., Cell-permeating α-ketoglutarate derivatives alleviate pseudohypoxia in succinate dehydrogenase-deficient cells. Mol Cell Biol 2007; 27:3282-9. As such, reducing α-KG by glutaminase inhibition could hypothetically enhance pseudohypoxia and favor tumor growth.

### III. METABOLIC INHIBITORS AGAINST AML CELLS HAVING IDH1 MUTATIONS

Morbidity and mortality from AML remains high. Patients afflicted with AML who are younger than 55 years of age and have no adverse prognostic features have approximately 50% long-term survival with conventional chemotherapies. Patients afflicted with AML who are older than 65 years of age and are treated with standard chemotherapy, however, have a median survival of 6 months, and only 7% remain in remission at three years.

The prevalence of the somatic mutations in IDH1 and IDH2 in de novo AML ranges between 10-15%. In AML, these mutations are strongly associated with intermediate risk cytogenetically normal AML and nucleophosmin 1 (NPM-1) mutation. IDH mutation status is an unfavorable prognostic factor for AML patients. Inhibiting glutaminase in the glutamine-addicted mIDH cells provides a selective and targeted, hence theoretically less toxic, approach to be incorporated in the current induction and consolidation chemotherapy regimens. Further, glutaminase inhibitors can be used as a maintenance therapy, particularly in prevalent and mostly incurable leukemia in elderly patients.

In addition to de novo AML, a relatively high incidence of IDH mutations (up to 20%) have been reported in the blast-phase of myeloproliferative neoplasm (MPN), as well as chronic-phase of primary myelofibrosis (PMF), regardless of JAK2 mutational status, indicating that although IDH mutations may arise early in the disease course, they are generally associated with leukemic transformation MPN.

Furthermore, mutations in IDH1 or IDH2 genes have been identified in 5% of myelodysplastic syndrome (MDS), in 9% of MDS/MPN, and in 10% of secondary AML cases. In patients with isolated del(5q), IDH mutation was significantly higher in high-risk MDS or AML as compared with those with low-risk MDS (22% vs. 0%).

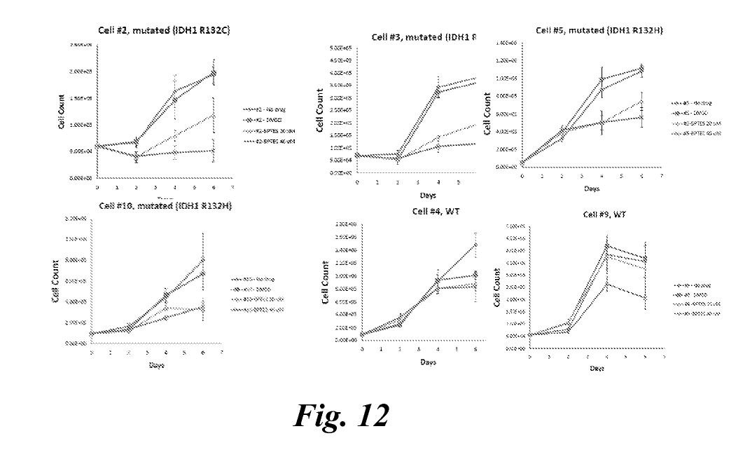

The presently disclosed data (see, for example, FIGS. 12A-12F) suggest that targeting glutaminase in mIDH has strong potential in other myeloid malignancies, such as MDS or MPN. This personalized targeted strategy also has a potential value in cancer prevention by thwarting leukemic transformation from more chronic phases.

More particularly, in some embodiments, the presently disclosed subject matter demonstrates that inhibition of glutaminase, for example, by compounds of Formula (I), e.g., bis-2-[5-(phenylacetamido)-1,3,4-thiadiazol-2-yl]ethyl sulfide (BPTES), and other glutaminase inhibitors disclosed herein, selectively kills primary acute myelogenous leukemia (AML) cells having mutant IDH1 (mIDH1) versus AML cells having the wild-type enzyme. Accordingly, the presently disclosed subject matter links the genotype of AML to susceptibility to glutaminase inhibitors. In this way, the presently disclosed subject matter potentiates the personalization of cancer medicine by connecting the genotype of AML with respect to IDH mutational status to sensitivity to cell killing by glutaminase inhibition with one or more of the presently disclosed glutaminase inhibitors, e.g., BPTES.

Although IDH mutations are frequently found in AML, a therapeutic strategy selective for the mutation has not been documented. The presently disclosed subject matter takes advantage of the biochemistry associated with the neo-function of mIDH to kill AML cells bearing the mutation with BPTES, which inhibits glutaminase. Acute myelogenous leukemia and other hematological conditions including, but not limited to, myeloproliferative disorders (MPD) transforming to leukemia, can have IDH mutations. IDH mutations increases 2-hydroxyglutarate (2HG) and hence can be diagnosed with serum 2-HG levels and DNA diagnostics. The neoplastic cells in patients with high 2-HG levels depend on glutamine to produce α-ketoglutarate for the mutant IDH neo-enzymatic activity. Depletion of α-ketoglutarate by glutaminase inhibition with BPTES will selectively kill leukemic cells with IDH mutations.

Accordingly, in some embodiments, the presently disclosed subject matter provides a method for stratifying patients afflicted with AML or transforming MPD (tMPD) by measuring serum 2-HG levels and/or DNA diagnosis for IDH1 or IDH2 mutation. Such stratification will allow for the selection of patients sensitive to therapy using a glutaminase inhibitor, for example, a compound of Formula (I), e.g., BPTES, and/or any glutaminase inhibitor known in the art, including those glutaminase inhibitors disclosed herein.

Accordingly, the presently disclosed subject matter provides methods that are useful for the classification and/or stratification of a subject or patient population. In one embodiment, for example, such stratification can be achieved by identification in a subject or patient population of one or more distinct profiles of at least one indicator (or co-indicator) of IDH1 or IDH2 mutation. In some embodiments, the indicator of IDH1 or IDH2 mutation is an elevated level of 2-HG measured in a biological sample from the subject. In particular embodiments, the biological sample is serum. Correlation of one or more traits in a subject with at least one indicator (or co-indicator) of IDH1 or IDH2 mutation can be used to gauge the subject's responsiveness to, or the efficacy of, a therapeutic treatment with a glutaminase inhibitor, which, in some embodiments, is a compound of Formula (I), e.g., BPTES, or other glutaminase inhibitor disclosed herein.

In other embodiments, the presently disclosed subject matter provides a method for monitoring the treatment of AML or tMPD patients with a glutaminase inhibitor by measuring serum 2-HG levels. Accordingly, in some embodiments, the presently disclosed methods can be used to diagnose, for the prognosis, or the monitoring of a disease state or condition. As used herein, the term “diagnosis” refers to a predictive process in which the presence, absence, severity or course of treatment of a disease, disorder or other medical condition is assessed. For purposes herein, diagnosis also includes predictive processes for determining the outcome resulting from a treatment. Likewise, the term “diagnosing,” refers to the determination of whether a subject exhibits one or more characteristics of a condition or disease. The term “diagnosing” includes establishing the presence or absence of, for example, a target antigen or reagent bound targets, or establishing, or otherwise determining one or more characteristics of a condition or disease, including type, grade, stage, or similar conditions. As used herein, the term “diagnosing” can include distinguishing one form of a disease from another. The term “diagnosing” encompasses the initial diagnosis or detection, prognosis, and monitoring of a condition or disease.

The term “monitoring,” such as in “monitoring the course of a disease or condition,” refers to the ongoing diagnosis of samples obtained from a subject having or suspected of having a disease or condition.

The term “prognosis,” and derivations thereof, refers to the determination or prediction of the course of a disease or condition. The course of a disease or condition can be determined, for example, based on life expectancy or quality of life. “Prognosis” includes the determination of the time course of a disease or condition, with or without a treatment or treatments. In the instance where treatment(s) are contemplated, the prognosis includes determining the efficacy of a treatment for a disease or condition.

As used herein, the term “risk” refers to a predictive process in which the probability of a particular outcome is assessed. The term “marker” refers to a molecule, such as a protein, including an antigen, that when detected in a sample is characteristic of or indicates the presence of a disease or condition or a risk of developing a disease or condition.

In yet other embodiments, the presently disclosed subject matter provides a method of using 2-HG serum levels in patients having MPD to detect transformation and provide early intervention with glutaminase inhibitor therapy.

In other embodiments, the presently disclosed subject matter provides the use of combination therapy (antimetabolites and anthracyclines) with a glutaminase inhibitor, e.g., a compound of Formula (I) or other glutaminase inhibitors disclosed herein, in AML or MPD with IDH mutations. It is thought that such combination therapy should provide more sustained remission of those conditions.

As used herein, an “antimetabolite” is a substance that competes with, replaces, or inhibits a specific metabolite of a cell and thereby interferes with the cell's normal metabolic functioning. An antimetabolite often is similar in structure to the metabolite, or enzymatic substrate, which is normally recognized and acted upon by an enzyme to form a substance required by the cell. Although the antimetabolite may resemble the substrate enough to be taken up by the cell, it does not react in the same way with the enzyme—either the enzymatic reaction is inhibited or the antimetabolite is converted by the enzyme into an aberrant component. An antimetabolite can have a toxic effect on a cell, such as halting cell growth and cell division. As such, antimetabolites can be used in chemotherapy for treating cancer.

When used in cancer treatment, antimetabolites interfere with DNA production and therefore cell division and tumor growth. Antimetabolites can mimic a purine, e.g., azathioprine or mercaptopurine, or a pyrimidine. Certain antimetabolites are purine analogues or pyrimidine analogues.

Representative purine analogue antimetabolites suitable for use with the presently disclosed subject matter include, but are not limited to, azathioprine, mercaptopurine, thioguanine, fludarabine, pentostatin, and cladribine. Representative pyrimidine analogues suitable for use with the presently disclosed subject matter include, but are not limited to, 5-fluorouracil SFU), floxuridine (FUDR), and cytosine arabinoside (cytarabine). The presence of an antimetabolite in a cell can prevent purines and/or pyrimidines from becoming incorporated into DNA, thereby stopping normal development and division.

As used herein, the term “anthracycline” includes a class of drugs used in chemotherapy that are derived from Streptomyces bacteria. Such compounds are used to treat a wide range of cancers, including leukemias, lymphomas, and breast, uterine, ovarian, and lung cancers. Representative anthracyclines suitable for use with the presently disclosed subject matter include, but are not limited to, daunorubicin, doxorubicin, epirubicin, idarubicin, valrubicin, mitoxantrone (an anthracycline analogue).

In embodiments wherein a glutaminase inhibitor is administered in combination with an antimetabolite and/or an anthracycline, the timing of administration of the glutaminase inhibitor and the antimetabolite and/or anthracycline can be varied so long as the beneficial effects of the combination of these agents are achieved. As such, the phrase “in combination with” refers to the administration of a presently disclosed glutaminase inhibitor with an antimetabolite and/or anthracycline either simultaneously, sequentially, or a combination thereof. Therefore, a cell or a subject administered a combination therapy can receive a presently disclosed glutaminase inhibitor and an antimetabolite and/or anthracycline at the same time (i.e., simultaneously) or at different times (i.e., sequentially, in either order, on the same day or on different days), so long as the effect of the combination of both agents is achieved in the cell or the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the presently disclosed glutaminase inhibitor and the antimetabolite and/or anthracycline are administered simultaneously, they can be administered to the cell or administered to the subject as separate pharmaceutical compositions, each comprising either a presently disclosed glutaminase inhibitor or an antimetabolite and/or anthracycline, or they can contact the cell as a single composition or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In some embodiments, when administered in combination, the agents have a synergistic effect.

In further embodiments, associated FLT3 mutations in AMLs with IDH mutations provide another level of stratification for combination therapy with FLT3 and glutaminase inhibitors.

### IV. DEFINITIONS

With respect to compounds of Formula (I), the terms below have the following meanings unless indicated otherwise.

“Alkyl” refers to a fully saturated acyclic monovalent radical containing carbon and hydrogen, which may be branched or a straight chain. Examples of alkyl groups are methyl, ethyl, n-butyl, n-heptyl, and isopropyl. “Lower alkyl,” a subset of this class, refers to alkyl having one to six carbon atoms, and more preferably one to four carbon atoms.

“Aralkyl” refers to a monovalent alkyl radical substituted with an aryl group, as defined herein, e.g. a benzyl group (—CH₂C₆H₅).

As used herein, “aryl” refers to a monocyclic aromatic or heteroaromatic group, having ring atoms selected from the group consisting of carbon, nitrogen, oxygen, and sulfur, and having at most three non-carbon ring atoms. The aryl group may be unsubstituted, or it may be substituted with one or more substituents selected from halogen, lower alkyl, lower alkoxy, amino, lower alkyl amino, amino(lower alkyl), and halo(lower alkyl). Preferably, each ring has at most three substituents, more preferably at most two, and most preferably one or no substituents.

A “pharmaceutically acceptable salt” of a compound described herein refers to the compound in protonated form with one or more anionic counterions, such as chloride, sulfate, phosphate, acetate, succinate, citrate, lactate, maleate, fumarate, palmitate, cholate, glutamate, glutarate, tartrate, stearate, salicylate, methanesulfonate, benzenesulfonate, sorbate, picrate, benzoate, cinnamate, and the like. Hydrochloride salts are a preferred group. The term also encompasses carboxylate salts having organic and inorganic cations, such as alkali and alkaline earth metal cations (for example, lithium, sodium, potassium, magnesium, barium and calcium); ammonium; or organic cations, for example, dibenzylammonium, benzylammonium, 2-hydroxyethylammonium, bis(2-hydroxyethyl) ammonium, phenylethylbenzylammonium, dibenzylethylenediammonium, and the like. Such salts may be formed by substitution of ionizable groups onto, for example, phenyl rings in group R₁ or R₂, which can be useful for increasing solubility or for reducing membrane permeability, if desired.

The presently disclosed compounds may exist in other forms depending on solvent, pH, temperature, and other variables known to practitioners skilled in the art. For example, equilibrium forms may include tautomeric forms. The compounds may be chemically modified to enhance specific biological properties, such as biological penetration, solubility, oral availability, stability, metabolism, or excretion. The compounds may also be modified to prodrug forms, such that the active moiety results from the action of metabolic or biochemical processes on the prodrug.

The term “subject” refers to an organism, tissue, or cell. A subject can include a human subject for medical purposes, such as diagnosis and/or treatment of an existing condition or disease or the prophylactic treatment for preventing the onset of a condition or disease, or an animal subject for medical, veterinary purposes, or developmental purposes. A subject also can include sample material from tissue culture, cell culture, organ replication, stem cell production and the like. Suitable animal subjects include mammals and avians. The term “mammal” as used herein includes, but is not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. The term “avian” as used herein includes, but is not limited to, chickens, ducks, geese, quail, turkeys, and pheasants. Preferably, the subject is a mammal or a mammalian cell. More preferably, the subject is a human or a human cell. Human subjects include, but are not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. A subject also can refer to cells or collections of cells in laboratory or bioprocessing culture in tests for viability, differentiation, marker production, expression, and the like.

“Effective amount”: In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, and the like.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Methods and Materials

Standard techniques were used to introduce the R132H mutation into human IDH1 engineered with a 6×-His-tag and produce lentivirus containing either WT or mutant IDH1. D54 cells or transformed normal human astrocytes (TNA) were transduced with virus, and lines were grown from individual cells using limiting dilution. IDH1 expression in response to 0.04 mg/mL doxycycline was confirmed using a 6×-His tag antibody (Millipore). Liquid chromatography/mass spectrometry was conducted as previously described. Dang L., et al., Cancer-associated IDH1 mutations produce 2-hydroxyglutarate. Nature 2009; 462:739-44. siRNA was used to knock down glutaminase, and cells were assessed for knockdown using Western blotting. Gao P., et al., c-Myc suppression of miR-23a/b enhances mitochondria/glutaminase expression and glutamine metabolism. Nature 2009; 458:762-5. Cell growth assays using alamarBlue were carried out as described following DMSO or bis-2-(5-phenylacetamido-1,2,4-thiadiazol-2-yl)ethyl sulfide (BPTES) treatment. Liolome W., et al., Glioblastoma cell growth is suppressed by disruption of Fibroblast Growth Factor pathway signaling. J Neurooncol 2009:94:359-66. Glutaminase activity was measured after treatment with DMSO or BPTES by incubating cell extracts with [3H]-glutamine. [3H]-glutamate was isolated from the reaction mixture using anion exchange and measured using a scintillation counter. All effects of BPTES were assayed 48 hours after treatment. Data were evaluated using a two-tailed Student's t-test. P≦0.05 was considered significant.

### Example 2

### Lentiviral Production

The open reading frame of IDH1 was PCR amplified from a full- length cDNA clone (Open Biosystems, clone ID: 30528912) to add a 6×-His-tag and restriction sites and cloned into pCR2.1. R132H (G395A) mutation was introduced using QuikChangeII Site-Directed Mutagenesis Kit (Stratagene) and confirmed via sequencing. WT and R132H IDH1 were cloned into pLVCT-tTR-KRAB (Addgene 11643 (Szulc J., et al., A versatile tool for conditional gene expression and knockdown. Nature Methods 2006; 3(2):109-16) using BamHI and EcoRV/SmaI or Duet011 (Addgene plasmid 17627, (Zhou B. Y., et al., Inducible and reversible transgene expression in human stem cells after efficient and stable gene transfer. Stem Cells 2007; 25(3):779-89) using BamHI and XhoI. The lentiviral vector was packaged using HEK 293T cells, psPAX2 (Addgene 12260, Didier Trono, packaging vector), and pMD2.G (Addgene 12259, Didier Trono, envelope plasmid).

### Example 3

### Stable Cell Lines

D54 or TNA cells were transduced with virus containing WT or R132H IDH1. D54 cells were transduced using virus containing pLVCT-tTR-KRAB constructs, and TNA were transduced with virus containing Duet011 constructs. Lines were grown from individual cells using limiting dilution and tested for IDH1 expression. Expression was confirmed using Western blot and 6×-His-tag antibodies (Millipore). 0.04 μg/mL doxycycline was used to induce expression for D54 lines. Cells were maintained in DMEM, 10% FBS, 4.5 g/L glucose, and penicillin/streptomycin. Transformed normal human astrocytes (TNA) were a gift from Christopher M. Counter.

### Example 4

### IDH1 WT Enzyme Activity Assay

D54 cells were seeded in a T75 flask at 5×105 cells, and IDH1 expression was induced with doxycycline 48 hrs before assaying. Cells were collected and resuspended in PBS, 0.1% Triton X-100, and Halt-Protease Inhibitor (Pierce). Cells were lysed for 30 min on ice and centrifuged for 30 min at 12000 rpm at 4° C. Protein concentration was measured using the BCA Assay (Pierce). Varying amounts of protein were added to IDH activity assay buffer (33 mM Tris, pH 7.6, 0.33 mM EDTA, 0.1 mM NADP+, 1.33 mM MnCl₂, and 1.3 mM isocitrate), and changes in absorbance at 340 nm after 5 min was documented for each protein amount. Yan H., et al., IDH1 and IDH2 mutations in gliomas. N Engl J Med 2009; 360(8):765-73.

### Example 5

### LC/MS

3×10⁵ (untreated) or 7.5×10⁵ (BPTES-treated) cells were seeded in a T75 flask. 24 hrs post-plating, media on the cells was changed to 4.5 g/L glucose, 10% HyClone dialyzed FBS (ThermoScientific), penicillin/streptomycin, 4 mM glutamine and 0.04 μg/mL doxycycline to induce IDH1 expression. 48 hrs post-plating, media was changed to include 10 μM BPTES or DMSO. Metabolites were measured in one of two ways: using a reverse phase column with tributylamine as an ion pairing agent, Luo B., et al., Simultaneous determination of multiple intracellular metabolites in glycolysis, pentose phosphate pathway and tricarboxylic acid cycle by liquid chromatography-mass spectrometry. J Chromatogr 2007; 1147(2):153-64, coupled to a Thermo Exactive Mass Spectrometer operating at 100,000 resolving power using a mass range of ±5 ppm or single reaction monitoring on a Thermo TSQ Quantum Triple Quadrupole Mass Spectrometer coupled to an aminopropyl HPLC column. Bajad S. U., et al., Separation and quantitation of water soluble cellular metabolites by hydrophilic interaction chromatography-tandem mass spectrometry. J Chromatogr 2006; 1125(1):76-88. Signals were normalized to cell number prior to data analysis.

### Example 6

### Glutaminase Knockdown

ON-TARGET plus SMARTpool siRNA against glutaminase and ONTARGET plus Non-targeting siRNA #2 were used. Cells were plated at a density of 1×10⁵ cells in a 6-well plate. 24 hrs after plating, the cells were induced with doxycycline. 48 hrs after plating, 100 nM of siRNA was transfected using oligofectamine (Invitrogen). For each time point, two wells were trypsinized, and cells were counted using trypan blue exclusion. Cells were assessed for knockdown using Western for glutaminase at 48 hrs. Gao P., et al., c-Myc suppression of miR-23a/b enhances mitochondrial glutaminase expression and glutamine metabolism. Nature 2009; 458(7239):762-5.

### Example 7

### Cell Proliferation

Cell growth assays using alamarBlue were carried out as described. Loilome W., et al., Glioblastoma cell growth is suppressed by disruption of Fibroblast Growth Factor pathway signaling. J Neurooncol 2009; 94(3):359-66. Cells were plated at a density of 500 cells/well in a 96-well black clear bottom plate (Becton Dickinson). At 24 hrs, media was changed to the appropriate media (DMEM with 4.5 g/L, 1.5 g/L or 0.1 g/L glucose, 10% FBS, penicillin/streptomycin, and 4 mM glutamine with or without doxycyline. 48 hours after plating, compounds or DMSO were added. Media and alamarBlue (Invitrogen) was added to a volume of 200 μL in each well. Fluorescence was measured at 48 hrs (AOA, BPTES) or 72 hrs (EGCG) using a Victor3 plate-reader (Perkin Elmer).

### Example 8

### Caspase 3/7 Activation Assay

Cells were plated at a density of 5000 cells/well in a 96-well black clear bottom plate (Becton Dickinson). 24 hrs after plating, media was changed to fresh media with or without 0.04 μg/mL doxycycline. 48 hrs after plating, 10 μM BPTES or DMSO was added to the well. Cells were incubated with BPTES for 24 hrs and assayed using Caspase-Glo 3/7 (Promega) according to manufacturer's instructions.

### Example 9

### Annexin V Staining

Cells were plated at a density of 2×10⁵ cells/well in a 6-well plate. Media was changed to media with or without doxycyline 24 hrs after plating. 48 hrs after plating, DMSO or 10-μM BPTES was added. Cells were incubated with BPTES for 24 hrs, stained using the FITC-Annexin V Apoptosis Detection Kit I (BD Pharmingen) according to manufacturer's instructions and analyzed via flow cytometry.

### Example 10

### Glutaminase Activity

2.5×10⁵ cells were plated in a 10-cm dish. 24 hrs post-plating, media was changed to 4.5 g/L glucose, 10% FBS, penicillin/streptomycin, and 4 mM glutamine with doxycyline. 48 hours after plating, compounds or DMSO were added. Cells were treated for 48 hrs, collected, and counted. Equal numbers of cells were pelleted and reconstituted in 100 μL of assay buffer (potassium phosphate, pH 8.2, 45 mM, protease inhibitors) and sonicated. Cell homogenate was incubated with [3H]-glutamine (91 nCi in 20 μL) for 45 min at room temperature. The reaction was terminated with imidazole buffer (pH 7, 20 mM, 100 μL) and passed through an anion exchange minicolumn (AG 1-X2 resin, chloride form, Bio Rad). The column was washed with imidazole (400 μL), and glutamate was eluted with HCl (0.1 N, 400 μL). An aliquot of the acid elution was measured using a scintillation counter.

### Example 11

### Glutamine Deprivation

24 hrs after plating 1,000 cells/well in 96-well plates, media was changed to DMEM with 1.5 g/L glucose, 10% HyClone dialyzed FBS, 4 mM glutamine, and penicillin/streptomycin with or without doxycycline. 48 hrs after plating, media was changed to DMEM with 1.5 g/L glucose, 10% dialyzed FBS, penicillin/streptomycin, with or without doxycycline at the indicated glutamine concentration. At 96 hrs, cell viability was assayed using Cell Counting Kit-8 (Enzo Life Sciences).

### Example 12

### Statistical Analyses

Data was evaluated using a two-tailed student's t-test. A p-value of ≦0.05 was considered significant. The heat map showing metabolite was clustered using the dist function in the R statics package.

### Example 13

### Materials And Methods Evaluation of the Effect of Glutaminase Inhibitors on Primary AML Cells with and without IDH1/2 Mutations

Primary AML cells were cultured in RPMI-1640 media with 20% fetal bovine serum (FBS), 20% 5637 cell-conditioned media and 1% antibiotics as previously described (Miyauchi et al., 1987). Growth curves were generated by manual counting of viable cells as assessed by trypan blue exclusion on a hemocytometer. The detail of the procedure is as following:

(1) The frozen cells are thawed and added into 10 mL of the media in a tissue culture flask and grown in a humidified incubator at 37° C. and 5% CO₂ for 4-5 days. Cells were counted every 24-48 h to estimate the cells needed for the experiment.

(2) When ready to plate the cells, FIRST, the desired media (alone or containing the inhibitor(s)) should be prepared and added into a 24-well plate [Use 1000 mL pipette]: (a) For no drug control, 1 mL per well of the media was added into each well of a 24-well plate; (b) For DMSO control, 0.1% DMSO concentration (1 μL DMSO per 1 mL of media) was prepared, and 1 mL per well was added; (c) For BPTES, a stock of 40 mM BPTES in DMSO was used. Then, 10-20 mL media containing each desired concentration of the drug were prepared. For 20 μM BPTES, 5 μL of the stock was added into 10 mL of the media. For 40 μM BPTES, 10 μL of the stock was added into 10 mL of the media. By doing this, the concentration of DMSO is constant and not more than 0.1%. BPTES is precipitated in PBS, so avoid dilution with PBS. By doing this the drug is dispersed in stock media before the media are dispensed to the cells; and (d) Before adding the above media into the 24-well plate, the media containing the drugs should be shaken or vortexed very well to assure equal distribution (homogenized) of the drugs or solvent in the media.

(3) Then, the media containing the vehicle (DMSO) or different concentrations of BPTES was transferred into a 24-well plate.

(4) In the next step, cells were spun down and the supernatant was discarded. Into each tube containing the cell pellet, 2-3 mL of PBS was added and mixed well by gentle pipetting. When the cells were well homogenized, they were counted by 1:1 trypan blue exclusion with hemocytometer.

(5) Then, 80,000-120,000 cells were added into the each well that already contained media. The starting number for each cell line among control/DMSO/drug must be the same and is calculated based on the count from the cell-containing PBS solution. If cells need more dilution, before adding them into the media, they should be recounted. To avoid the shear effect, use a large size pipette. Gently pipette up and down once or twice after adding the cells into the media. Keep the 24-well plates in a humidified incubator at 37° C. and 5% CO₂.

(6) Cells were counted every other day using a hemocytometer after staining with trypan blue (1:1). Before each count, cells were gently mixed by pipetting. Each time 20 μL of the cell-containing media was mixed with 20 μL trypan blue. Primary AML cells are small-sized; special attention should be paid to differentiate them from cellular debris or other artifacts. Cells were counted in the 4 outer squares of the hemocytometer, and the number was divided by 2 (=n). The concentration is n×10⁴ cells/mL. When cell concentration was low, more grids were counted and the cell concentrations were calculated accordingly.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Abbas S, Lugthart S, Kavelaars F G, et al. Acquired mutations in the genes encoding IDH1 and IDH2 both are recurrent aberrations in acute myeloid leukemia (AML): prevalence and prognostic value. Blood 2010.
Aghili M, Zahedi F, Rafiee E. Hydroxyglutaric aciduria and malignant brain tumor: a case report and literature review. J Neurooncol 2009; 91:233-6.
Alonso F J, Segura J A, Lora J, et al. Sensitisation of Ehrlich ascitic tumour cells to methotrexate by inhibiting glutaminase. Anticancer Res 2005; 25:3315-20.
Bajad S U, Lu W, Kimball E H, Yuan J, Peterson C, Rabinowitz J D. Separation and quantitation of water soluble cellular metabolites by hydrophilic interaction chromatography-tandem mass spectrometry. J Chromatogr 2006; 1125(1):76-88.
Bleeker F E, Atai N A, Lamba S, et al. The prognostic IDH1 (R132) mutation is associated with reduced NADP+-dependent IDH activity in glioblastoma. Acta Neuropathol 2010; 119:487-94.
Chou W C, Hou H A, Chen C Y, et al. Distinct clinical and biologic characteristics in adult acute myeloid leukemia bearing the isocitrate dehydrogenase 1 mutation. Blood 2010; 115:2749-54.
Dang C V. Rethinking the Warburg effect with Myc micromanaging glutamine metabolism. Cancer Res; 70:859-62.
Dang L, White D W, Gross S, et al. Cancer-associated IDH1 mutations produce 2-hydroxyglutarate. Nature 2009; 462:739-44.
DeBerardinis R J, Cheng T. Q's next: the diverse functions of glutamine in metabolism, cell biology and cancer. Oncogene 2010; 29:313-24.
DeBerardinis R J, Mancuso A, Daikhin E, et al. Beyond aerobic glycolysis: transformed cells can engage in glutamine metabolism that exceeds the requirement for protein and nucleotide synthesis. Proc Natl Acad Sci USA 2007; 104:19345-50. Gao P, Tchernyshyov I, Chang T C, et al. c-Myc suppression of miR-23a/b enhances mitochondrial glutaminase expression and glutamine metabolism. Nature 2009; 458:762-5.
Gross S, Cairns R A, Minden M D, et al. Cancer-associated metabolite 2-hydroxyglutarate accumulates in acute myelogenous leukemia with isocitrate dehydrogenase 1 and 2 mutations. J Exp Med 2010; 207:339-44.
Hartmann C, Meyer J, Balss J, et al. Type and frequency of IDH1 and IDH2 mutations are related to astrocytic and oligodendroglial differentiation and age: a study of 1,010 diffuse gliomas. Acta Neuropathol 2009; 118:469-74.
Kim J W, Dang C V. Cancer's molecular sweet tooth and the Warburg effect. Cancer Res 2006; 66:8927-30.
Latini A, Scussiato K, Rosa R B, et al. D-2-hydroxyglutaric acid induces five stress in cerebral cortex of young rats, Eur J Neurosci 2003; 17:2017-22.
Latini A, da Silva C G, Ferreira G C, et al. Mitochondrial energy metabolism is markedly impaired by D-2-hydroxyglutaric acid in rat tissues. Mol Genet Metab 2005; 86:188-99.
Lobo C, Ruiz-Bellido M A, Aledo J C, Marquez J, Nunez De Castro I, Alonso F J. Inhibition of glutaminase expression by antisense mRNA decreases growth and tumourigenicity of tumour cells. Biochem J 2000; 348 Pt 2:257-61.
Lora J, Alonso F J, Segura J A, Lobo C, Marquez J, Mates J M. Antisense glutaminase inhibition decreases glutathione antioxidant capacity and increases apoptosis in Ehrlich ascitic tumour cells. Eur J Biochem 2004; 271:4298-306.
Loilome W, Joshi A D, ap Rhys C M, et al. Glioblastoma cell growth is suppressed by disruption of Fibroblast Growth Factor pathway signaling. J Neurooncol 2009; 94: 359-66.
Luo B, Groenke K, Takors R, Wandrey C, Oldiges M. Simultaneous determination of multiple intracellular metabolites in glycolysis, pentose phosphate pathway and tricarboxylic acid cycle by liquid chromatography-mass spectrometry. J Chromatogr 2007; 1147(2):153-64.
MacKenzie E D, Selak M A, Tennant D A, et al, Cell permeating α-ketoglutarate derivatives alleviate pseudohypoxia in succinate dehydrogenase-deficient cells. Mol Cell Biol 2007; 27:3282-9.
Marcucci G, Maharry K, Wu Y Z, et al. IDH1 and IDH2 gene mutations identify novel molecular subsets within de novo cytogenetically normal acute myeloid leukemia: a Cancer and Leukemia Group B study. J Clin Oncol 2010; 28:2348-55.
Mardis E R, Ding L, Dooling D J, et al. Recurring mutations found by sequencing an acute myeloid leukemia genome. N Engl J Med 2009; 361:1058-66.
Medina M A. Glutamine and cancer. J Nutr 2001; 131:2539-42S.
Miyauchi J, Kelleher C A, Yang Y C, et al. The effects of three recombinant growth factors, IL-3, GM-CSF, and G-CSF, on the blast cells of acute myeloblastic leukemia maintained in short-term suspension culture. Blood 1987; 70:657-63.
Moreadith R W, Lehninger A L. The pathways of glutamate and glutamine oxidation by tumor cell mitochondria. Role of mitochondrial NAD(P)+-dependent malic enzyme. J Biol Chem 1984; 259:6215-21.
Parsons D W, Jones S. Zhang X, et al. An integrated genomic analysis of human glioblastoma multiforme. Science 2008; 321:1807-12.
Robinson M M, McBryant S J, Tsukamoto T, et al. Novel mechanism inhibition of rat kidney-type glutaminase bis-2-(5-phenylacetamido-1,2,4-thiadiazol-2-yl)ethyl sulfide (BPTES). Biochem J 2007; 406:407-14.
Rzem R. Vincent M F, Van Schaftingen E, Veiga-da-Cunha M. L-2-hydroxyglutaric aciduria, a defect of metabolite repair. J Inherit Metab Dis 2007:30:681-9.
Struys E A, Salomons G S, Achouri Y, et al. Mutations in the D-2-hydroxyglutarate dehydrogenase gene cause D-2-hydroxyglutaric aciduria. Am J Hum Genet. 2005; 76:358-60.
Szulc J, Wiznerowicz M, Sauvain M O, Trono D, Aebischer P. A versatile tool for conditional gene expression and knockdown. Nature Methods 2006; 3(2):109-16.
Tennant D A, Duran R V, Gottlieb E Targeting metabolic transformation for cancer therapy. Not Rev Cancer 2010; 10:267-77.
Ward P S, Patel J, Wise D R, et al. The common feature of leukemia-associated IDH1 and IDH2 mutations is a neomorphic enzyme activity converting α-ketoglutarate to 2-hydroxyglutarate. Cancer Cell 2010; 17:225-34.
Wise D R, DeBerardinis R J, Mancuso A, et al. Myc regulates a transcriptional program that stimulates mitochondrial glutaminolysis and leads to glutamine addiction. Proc Natl Acad Sci USA 2008; 105:18782-7.
Yan H, Parsons D W, Jin G, et al. IDH1 and IDH2 mutations in gliomas. N Engl J Med 2009; 360:765-73.
Zhao S, Lin Y, Xu W. et al. Glioma-derived mutations in IDH1 dominantly inhibit IDH1 catalytic activity and induce HIF-1α. Science 2009; 324:261-5.
Zhou B Y, Ye Z, Chen G, Gao Z P, Zhang Y A, Cheng L. Inducible and reversible transgene expression in human stem cells after efficient and stable gene transfer. Stem Cells 2007; 25(3):779-89.
Yan H, Parsons D W, Jin G, et al. IDH1 and IDH2 mutations in gliomas. N Engl J Med 2009; 360(8):765-73.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating a cancer having a mutant isocitrate dehydrogenase (IDH) in a subject in need of treatment thereof, the method comprising administering to the subject a therapeutically effective amount of a glutaminase inhibitor.

2. The method of claim 1, wherein the glutaminase inhibitor is selected from the group consisting of:
(a) a compound of Formula (I): 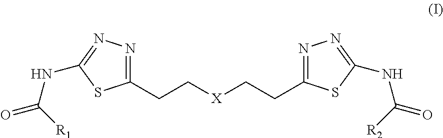
wherein:
X is sulfur or oxygen;
R₁ and R₂ are independently selected from the group consisting of lower alkyl, lower alkoxyl, aryl, thiophenyl and —(CH₂)ₙ-aryl;
wherein n is 0 or 1, and aryl is a monocyclic aromatic or heteroaromatic group, having ring atoms selected from the group consisting of carbon, nitrogen, oxygen, and sulfur, and having at most three non-carbon ring atoms, which group may be unsubstituted or substituted with one or more substituents selected from the group consisting of halogen, lower alkyl, lower alkoxyl, amino, lower alkyl amino, amino(lower alkyl), or halo(lower alkyl);
(b) a compound selected from the group consisting of 6-diazo-5-oxo-L-norleucine, acivicin, N-ethylmaleimide, p-chloromercuriphenylsulfonate, L-2-amino-4-oxo-5-chloropentoic acid, azaserine, and 5-(3-bromo-4-(dimethylamino)phenyl)-2,2-dimethyl-2,3,5,6-tetrahydrobenzo[a]phenanthridin-4(1H)-one; and
pharmaceutically acceptable salts thereof.

3. The method of claim 2, wherein the glutaminase inhibitor is a compound of Formula (I) and X is sulfur.

4. The method of claim 3, wherein each of R₁ and R₂ is —(CH₂)ₙ-aryl.

5. The method of claim 3, wherein each of R₁ and R₂ is 2-thiophenyl.

6. The method of claim 3, wherein each of R₁ and R₂ is 2-furanyl.

7. The method of claim 3, wherein each of R₁ and R₂ is phenyl or benzyl, unsubstituted or substituted with lower alkyl or lower alkoxyl.

8. The method of claim 7, wherein each of R₁ and R₂ is benzyl.

9. The method of claim 7, wherein each of R₁ and R₂ is p-methoxy phenyl.

10. The method of claim 7, wherein each of R₁ and R₂ is m-tolyl.

11. The method of claim 2, wherein the glutaminase inhibitor is a compound of Formula (I) and each of R₁ and R₂ is lower alkoxyl.

12. The method of claim 11, wherein each of R₁ and R₂ is ethoxy.

13. The method of claim 2, wherein the glutaminase inhibitor is a compound of Formula (I) and each of R₁ and R₂ is lower alkyl.

14. The method of claim 13, wherein each of R₁ and R₂ is t-butyl.

15. The method of claim 2, wherein the compound of Formula (I) is bis-2-(5-phenylacetamido-1,2,4-thiadiazol-2-yl)ethyl sulfide (BPTES):

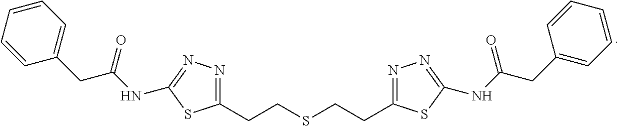 [SMILES: O=C(Cc1ccccc1)Nc1nnc(CCSCCc2nnc(NC(=O)Cc3ccccc3)s2)s1 ; "."]

16. The method of claim 1, wherein the mutant IDH is selected from the group consisting of mutant IDH1 and mutant IDH2.

17. The method of claim 1, wherein the cancer is selected from the group consisting of a malignant low-grade glioma, a secondary glioblastoma, a transforming myeloproliferative disorder (tMPD), and an acute myelogenous leukemia (AML).

18. The method of claim 1, further comprising administering the glutaminase inhibitor in combination with one or more of an antimetabolite, an anthracycline, and combinations thereof.

19. A method for selectively inhibiting growth of a cell expressing mutant isocitrate dehydrogenase (IDH), the method comprising administering to the cell an effective amount of a glutaminase inhibitor.

20. The method of claim 19, wherein the glutaminase inhibitor is selected from the group consisting of:
(a) a compound of Formula (I): 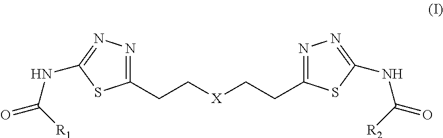
wherein:
X is sulfur or oxygen;
R₁ and R₂ are independently selected from the group consisting of lower alkyl, lower alkoxyl, aryl, thiophenyl and —(CH₂)ₙ-aryl;
wherein n is 0 or 1, and aryl is a monocyclic aromatic or heteroaromatic group, having ring atoms selected from the group consisting of carbon, nitrogen, oxygen, and sulfur, and having at most three non-carbon ring atoms, which group may be unsubstituted or substituted with one or more substituents selected from the group consisting of halogen, lower alkyl, lower alkoxyl, amino, lower alkyl amino, amino(lower alkyl), or halo(lower alkyl);
(b) a compound selected from the group consisting of 6-diazo-5-oxo-L-norleucine, acivicin, N-ethylmaleimide, p-chloromercuriphenylsulfonate, L-2-amino-4-oxo-5-chloropentoic acid, azaserine, and 5-(3-bromo-4-(dimethylamino)phenyl)-2,2-dimethyl-2,3,5,6-tetrahydrobenzo[a]phenanthridin-4(1H)-one; and
pharmaceutically acceptable salts thereof.

21. The method of claim 20, wherein the mutant IDH is selected from the group consisting of mutant IDH1 and mutant IDH2.

22. The method of claim 20, wherein the cell comprises a cancer cell.

23. The method of claim 22, wherein the cancer cell is selected from the group consisting of a malignant low-grade glioma, a secondary glioblastoma, and an acute myelogenous leukemia (AML).

24. A method for selecting a subject for treatment with a glutaminase inhibitor, the method comprising measuring a 2-hydroxyglutarate (2-HG) level in a biological sample from the subject and selecting the subject for treatment if the 2-HG level exceeds a predetermined threshold value.

25. The method of claim 24, wherein the biological sample comprises serum.

26. The method of claim 24, wherein the subject is afflicted with or is suspected of being afflicted with a cancer.

27. The method of claim 26, wherein the cancer is selected from the group consisting of a malignant low-grade glioma, a secondary glioblastoma, a transforming myeloproliferative disorder, and an acute myelogenous leukemia (AML).

28. The method of claim 24, further comprising stratifying the subject based on the 2-HG level in the biological sample.

29. A method for selecting a subject for treatment with a glutaminase inhibitor, the method comprising identifying the presence of a IDH1 or IDH2 mutation in a cell or tissue from the subject and selecting the subject for treatment with a glutaminase inhibitor.

30. The method of claim 29, wherein the method further comprises identifying the presence of an FLT3 mutation in the subject.

31. A method for monitoring a treatment of a subject with a glutaminase inhibitor, the method comprising measuring a 2-HG level in a biological sample from the subject and comparing the 2-HG level in the subject with a 2-HG level selected from the group consisting of a 2-HG level in a previous biological sample from the subject, a predetermined threshold value of 2-HG, a 2-HG level in one or more biological samples from a control group, and combinations thereof.

32. A method for detecting transformation in a subject afflicted with a myeloproliferative disorder (MPD), the method comprising measuring a 2-HG level in a biological sample from the subject and comparing the 2-HG level with a predetermined threshold value to detect transformation.

33. The method of claim 32 further comprising prophylactic treatment of the subject with a glutaminase inhibitor.

34. The method of claim 33, wherein the glutaminase inhibitor is selected from the group consisting of:
(a) compound of Formula (I): 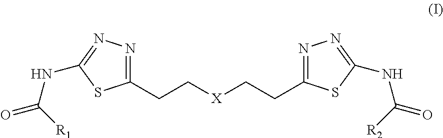
wherein:
X is sulfur or oxygen;
R₁ and R₂ are independently selected from the group consisting of lower alkyl, lower alkoxyl, aryl, thiophenyl and —(CH₂)ₙ-aryl;
wherein n is 0 or 1, and aryl is a monocyclic aromatic or heteroaromatic group, having ring atoms selected from the group consisting of carbon, nitrogen, oxygen, and sulfur, and having at most three non-carbon ring atoms, which group may be unsubstituted or substituted with one or more substituents selected from the group consisting of halogen, lower alkyl, lower alkoxyl, amino, lower alkyl amino, amino(lower alkyl), or halo(lower alkyl);
(b) a compound selected from the group consisting of 6-diazo-5-oxo-L-norleucine, acivicin, N-ethylmaleimide, p-chloromercuriphenylsulfonate, L-2-amino-4-oxo-5-chloropentoic acid, azaserine, and 5-(3-bromo-4-(dimethylamino)phenyl)-2,2-dimethyl-2,3,5,6-tetrahydrobenzo[a]phenanthridin-4(1H)-one; and
pharmaceutically acceptable salts thereof.
